(19) United States
(12) Patent Application Publication
(10) Pub. No.: US 2026/0133191 A1
(43) Pub. Date: May 14, 2026

(54) DETECTION OF AN AMPHIPHILE USING VISUAL INSPECTION OF A LIGAND-MODIFIED SUBSTRATE

(71) Applicant: Ohio State Innovation Foundation, Columbus, OH (US)

(72) Inventors: Xiaoguang WANG, Powell, OH (US); Yang XU, Columbus, OH (US)

(21) Appl. No.: 19/120,127

(22) PCT Filed: Oct. 5, 2023
(86) PCT No.: PCT/US2023/034543

§ 371 Date: Apr. 10, 2025

Related U.S. Application Data
(60) Provisional application No. 63/414,981, filed on Oct. 11, 2022.

(51) Int. Cl. G01N 33/543; G01N 21/29; G01N 33/569

(52) U.S. Cl. CPC G01N 33/54393; G01N 21/29; G01N 33/56911; G01N 2333/195

(57) ABSTRACT

Disclosed herein are compositions, devices, systems, and methods for detection of an amphiphile using visual inspection of a ligand-modified substrate. For example, disclosed herein are assays for detection of an amphiphile via visual inspection, the assay comprising a ligand-modified substrate and a film of lubricant disposed on the ligand-modified substrate. Also disclosed herein are methods of use of any of the assays disclosed herein. In some examples, the methods comprise contacting any of the assays disclosed herein with a liquid sample; tilting the assay; and visually inspecting the liquid sample disposed on the assay to determine a property of the liquid sample. In some examples, the amphiphile comprises a biological amphiphile, such as an endotoxin. In some examples, the biological amphiphile comprises an endotoxin secreted by a gram-negative bacteria, such as Escherichia coli.

### CROSS-REFERENCE TO RELATED APPLICATIONS

This application claims the benefit of priority to U.S. Provisional Application No. 63/414,981, filed Oct. 11, 2022, which is hereby incorporated herein by reference in its entirety.

### BACKGROUND

Biological amphiphiles can affect ecological and/or biological functions even at extremely low concentrations. Therefore, the detection of low amphiphile concentrations can be used to monitor the condition of such systems. Reliable and sensitive detection of various biological amphiphiles can provide important safety information, such as regarding water and food supplies. However, monitoring the concentration biological amphiphiles is conventionally achieved using assays that rely upon specialized chemicals and equipment, making these tests unsuitable for portable and fast in-situ or point-of-care amphiphile detection. Assays and methods that enable fast and reliable detection of biological amphiphiles and their concentration that do not require the use of specialized equipment are needed. The compositions, devices, methods, and systems discussed herein address these and other needs.

### SUMMARY

In accordance with the purposes of the disclosed compositions, devices, methods, and systems as embodied and broadly described herein, the disclosed subject matter relates to detection of an amphiphile using visual inspection of a ligand-modified substrate.

For example, disclosed herein are assays for detection of an amphiphile via visual inspection. The assays comprise a ligand-modified substrate comprising a substrate having a plurality of ligands attached to a surface thereof. The assays further comprise a lubricant disposed on the ligand-modified substrate, such that the lubricant forms a film on the ligand-modified substrate. The plurality of ligands comprises a first population of ligands and a second population of ligands, the first population of ligands being different than the second population of ligands.

In some examples, the substrate comprises silicon, glass, a polymer, a cellulosic substrate, a metal, or a combination thereof. In some examples, the substrate comprises glass, such as a glass slide. In some examples, the substrate comprises a flexible substrate. In some examples, the substrate comprises a rigid substrate.

In some examples, each of the plurality of ligands comprises a silyl, a thiol, a sulfate, a carboxylic acid, an amine, or a combination thereof. In some examples, each of the plurality of ligands comprises a silyl, such as a silane or an alkoxy silane.

In some examples, the first population of ligands have a first average length, the second populations of ligands have a second average length, and the first average length is different than the second average length.

In some examples, the lubricant comprises silicon oil.

In some examples, the amphiphile comprises a biological amphiphile. In some examples, the biological amphiphile comprises an endotoxin. In some examples, the biological amphiphile comprises an endotoxin secreted by a gram-negative bacteria. In some examples, the biological amphiphile comprises an endotoxin secreted by Escherichia coli.

In some examples, the assay comprises a plurality of domains, wherein each domain is different with respect to ligand functionalization (e.g., identity of ligand, density or concentration of ligands).

Also disclosed herein are methods of making any of the assays disclosed herein. In some examples, the methods comprise disposing the lubricant on the ligand-modified substrate. In some examples, the method further comprises making the ligand-modified substrate. In some examples, the method comprises contacting the substrate with a plurality of ligands having a functional group configured to covalently or ionically bond to the substrate.

Also disclosed herein are methods of use of any of the assays disclosed herein. In some examples, the methods comprise contacting any of the assays disclosed herein with a liquid sample; subsequently tilting the assay; and subsequently visually inspecting the liquid sample disposed on the assay to determine a property of the liquid sample.

In some examples, the liquid sample comprises a solvent. In some examples, the solvent comprises water.

In some examples, the method further comprises collecting the liquid sample.

In some examples, the method further comprises purifying the liquid sample before contacting the liquid sample with the assay.

In some examples, visually inspecting the liquid sample disposed on the assay comprises determining the angle at which the liquid sample starts to slide, the speed at which the liquid sample moves along the assay when tilted at a given angle, the distance that the liquid sample moves along the assay when tilted at a given angle.

In some examples, the property of the liquid sample comprises the presence of the amphiphile the liquid sample, the concentration of the amphiphile in the liquid sample, the identity of the amphiphile, or a combination thereof.

In some examples, the amphiphile comprises a biological amphiphile. In some examples, the biological amphiphile comprises an endotoxin. In some examples, the biological amphiphile comprises an endotoxin secreted by a gram-negative bacteria. In some examples, the biological amphiphile comprises an endotoxin secreted by Escherichia coli.

In some examples, the endotoxin is present in the liquid sample at a concentration of from 1 picogram/mL to 50 nanograms/mL. In some examples, the endotoxin is present in the liquid sample at a concentration of from 100 picograms/mL to 1 nanogram/mL.

In some examples, the liquid sample comprises a droplet.

In some examples, the liquid sample has a volume of from 1 microliter (μL) to 50 milliliters (mL).

In some examples, visually inspecting the liquid sample disposed on the assay comprises naked-eye detection.

In some examples, the method further comprises diagnosing and/or monitoring an infection with the biological amphiphile in a subject based on the property of the liquid sample. In some examples, the method further comprises selecting a course of therapy for the subject based on the property of the liquid sample.

Also disclosed herein are devices. For example, also disclosed herein are devices comprising: a receptacle configured to at least partially contain any of the assays disclosed herein; wherein, when the device is assembled together with a liquid sample, then the receptacle is configured to at least partially contain the assay in contact with the liquid sample.

In some examples, the device further comprises a means for tilting the assay to a desired angle.

In some examples, the device further comprises a detector configured to collect an optical signal from the liquid sample disposed on the assay. In some examples, detector comprises a camera configured to take a picture and/or video of the assay and/or liquid sample. In some examples, the camera is configured to take a picture and/or video of the assay before, after, and/or during any of the methods described herein, for example such that the visual inspection can be performed remotely in time and/or space.

In some examples, the device further comprises a computing device configured to receive and process the optical signal from the detector to determine a property of the liquid sample. In some examples, the device is further configured to output the property of the liquid sample and/or a feedback signal based on the property of the liquid sample. In some examples, the feedback signal comprises haptic feedback, auditory feedback, visual feedback, or a combination thereof.

In some examples, the device is a point-of-care device.

In some examples, the device is a handheld device.

In some examples, the device is a benchtop device.

In some examples, the device is a high-throughput device.

In some examples, the device is configured to analyze a plurality of liquid samples.

In some examples, the device is configured to perform any of the methods disclosed herein.

Additional advantages of the disclosed compositions, devices, systems, and methods will be set forth in part in the description which follows, and in part will be obvious from the description. The advantages of the disclosed compositions, devices, systems, and methods will be realized and attained by means of the elements and combinations particularly pointed out in the appended claims. It is to be understood that both the foregoing general description and the following detailed description are exemplary and explanatory only and are not restrictive of the disclosed compositions, devices, systems, and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. An illustration of the Si oil on the functionalized surface and a droplet thereon.

FIG. 2. Schematic illustration of sorting aqueous droplets with different concentrations.

FIG. 3. Initial substrate with droplets having varying concentration of SDS.

FIG. 4. Corresponds to FIG. 3 after tilting the substrate to vertical, showing substrate differentiates droplets with differing concentrations.

FIG. 5. Initial substrate with droplets having varying volumes.

FIG. 6. Corresponds to FIG. 5 after tilting the substrate to vertical, showing substrate differentiates droplets with differing volumes.

### DETAILED DESCRIPTION

The compositions, devices, methods, and systems described herein may be understood more readily by reference to the following detailed description of specific aspects of the disclosed subject matter and the Examples included therein.

Before the present compositions, devices, methods, and systems are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₂, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z‘ where Z’ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(=OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-pentoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The terms “amine” or “amino” as used herein are represented by the formula —NZ¹Z²Z³, where Z¹, Z², and Z can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

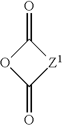

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N=N.

The term “carboxylic acid” as used herein is represented by the formula —C(O)OH.

A “carboxylate” or “carboxyl” group as used herein is represented by the formula —C(O)O⁻.

A “carbonate ester” group as used herein is represented by the formula Z¹OC(O)OZ².

The term “cyano” as used herein is represented by the formula —CN.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

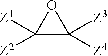

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein comprises the formula —S—.

The term “thiol” as used herein is represented by the formula —SH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Assays

Disclosed herein are compositions, devices, systems, and methods for detection of an amphiphile using visual inspection of a ligand-modified substrate.

For example, disclosed herein are assays for detection of an amphiphile via visual inspection, the assay comprising: a ligand-modified substrate comprising a substrate having a plurality of ligands attached to a surface thereof, and a lubricant disposed on the ligand-modified substrate, such that the lubricant forms a film on the ligand-modified substrate.

The substrate can be flexible or rigid. The substrate can comprise any suitable material. Examples of substrates include, but are not limited to, glass, quartz, silicon, silicon dioxide, nitrides (e.g., silicon nitride), polymers (e.g., polycarbonate, polydimethylsiloxane (PDMS)), a cellulosic substrate (e.g., wood, paper, etc.), metals, and combinations thereof. In some examples, the substrate comprises silicon, glass, a polymer, a cellulosic substrate, a metal, or a combination thereof. In some examples, the substrate comprises glass, such as a glass slide.

The plurality of ligands can comprise a first population of ligands and a second population of ligands, the first population of ligands being different than the second population of ligands. For example, the first population of ligands can have a first average length and the second populations of ligands can have a second average length, wherein the first average length is different than the second average length. The use of two or more different ligands can, for example, introduce nanoscopic or angstrom-level roughness to the ligand-modified substrate.

The plurality of ligands can, for example, the covalently or ionically bound to a surface of the substrate. In some examples, each of the plurality of ligands comprises a portion configured to interact with, and optionally bind to (e.g., electrostatically, ionically, or covalently), at least a portion of the amphiphile.

In some examples, each of the plurality of ligands comprises a silyl, a thiol, a sulfate, a carboxylic acid, an amine, or a combination thereof. In some examples, each of the plurality of ligands comprises a silyl, such as a silane or an alkoxy silane.

In some examples, the assay can comprise a plurality of domains, wherein each domain is different with respect to ligand functionalization (e.g., identity of ligand, density or concentration of ligands on surface, etc. or a combination thereof).

The lubricant can comprise any suitable material. For example, the lubricant can comprise a perfluorinated oil such as Krytox (a commercial product from Dupont Company), mineral oil, silicon oil, and the like. In some examples, the lubricant comprises silicon oil.

The amphiphile can comprise any suitable compound, such as a biological or synthetic amphiphile. Examples of synthetic amphiphiles include, but are not limited to, synthetic surfactant and detergents, anionic, cationic, nonionic and zwitterionic surfactants.

In some examples, the amphiphile comprises a biological amphiphile. The biological amphiphile can comprise any biomolecule with an aliphatic tail group (e.g., hydrocarbon tail), such as a phospholipid. In some examples, the biological amphiphile comprises an endotoxin. In some examples, the biological amphiphile comprises an endotoxin secreted by a gram-negative bacteria. As used herein, “gram-negative bacteria” refers to bacteria that turn red when Gram stain is applied to them. Gram-negative bacteria have a thin peptidoglycan layer as compared to gram-positive bacteria.

Examples of gram-negative bacteria include, but are not limited to, Acetic acid bacteria, Acidaminococcus, Acinetobacter baumannii, Acinetobacier guerrae, Acinetobacter portensis, Actinobacillus equuli, Agrobacterium tumefaciens, Akkermansia glycaniphila, Akkermansia muciniphila, Anaerobiospirillum, Anaerolinea thermolimosa, Anaerolinea thermophila, Arcobacter spp., Arcobacter skirrowii, Armatimonas rosea, Azotobacter salinestris, Bacteroides spp., Bacteroides caccae, Bacteroides fragilis, Bacteroides thetaiotaomicron, Bacteroides ureolyticus, Bacteroidota, Bartonella japonica, Bartonella koehlerae, Bartonella taylorii, Basfia succiniciproducens, Bdellovibrio, Bernardeliaceae, Borreliaceae, Brachyspira, Bradvrhizobium japonicum, Budviciaceae, Caldilinea aerophile, Cardiobacterium spp., Cardiobacterium hominis, Cesiribacteraceae, Chaperone-usher fimbriae, Chishuiella, Christensenella, Chthonomonas calidirosea, Coxiella burnetiid, Dehalogenimonas lykanthroporepellens, Desulfurobacterium atlamicur, Devosia pacifica. Devosia psychrophile, Devosia soli, Devosia subaequoris, Devosia submarina, Devosia yakushimensis, Dialister, Dicyoglomus thermophilur, Dinoroseobacter shibae. Ekhidna hutea, Enterobacter spp., Einerobacter cloacae, Enterobacter cowanii, Enterobacterales, Enterobacteriaceae, Erwiniaceae, Escherichia spp., Escherichia coli, Escherichia fergusonii, Escherichia hermannii, Fimbriimonas ginsengisoli, Flavobacterium spp., Flavobacterium akiainvivens, Fulvivirgaceae. Fusobacterium necrophorum, Fusobacterium nucleatum, Fusobacterium polymorphutm, Ghuconacetobacter diazotrophicus, Haemophilus felis, Haemophilus haemolyticus, Haemophilus influenzae, Haemophilus pittmaniae, Hafniaceae, Helicobacter spp., Helicobacter bizzozeronii, Helicobacter cetorum, Helicobacter cinaedi, Helicobacter heilmani s.s, Helicobacter heilmanuii sensu lato, Helicobacter salomonis, Helicobacter suis, Helicobacter typhlonius. Hymenobacteraceae, Kingella kingae, Klebsiella huaxiensis, Klebsiella pneumoniae, Kluyvera ascorbate, Kluyvera cryocrescens, Kozakia baliensis, Legionella spp., Legionella clemsonensis, Legionella pneumophia, Leptonema illini, Leptotrichia buccalis, Levilinea saccharolytica, Lewinellaceae, Luteimonas aestuarii, Luteimonas aquatica, Luteimonas composti, Luteimonas hntimaris, Luteimonas marina, Luteimonas mephitis, Luteimonas vadose, Maribacter spp., Marinmflexile, Megamonas, Megasphaera, Meiothermus spp., Meiothermus timidus, Methylobacterium fujisawaense, Methylomirabidis oxyfera, Microscillaceae, Mixta spp., Mixta calida, Moraxella spp., Moraxella bovis, Moraxella lacunata, Moraxella osloensis, Morganella morganii, Morganellaceae, Muricauda, Mycoplasma spumans, Neisseria cinerea, Neisseria gonorrhoeae, Neisseria meningitidis, Neisseria powiyaccharea, Neisseria sicca, Nitrosomonas eutropha, Nitrosomonas halophila, NWrosomonas stercoris, Nonpathogenic organisms, Pectinatus, Pectobacteriaceae, Pelosinus, Persicobacteraceae, Pluralibacter gergoviae, Pluraibacter pyrinus, Propionispora. Proteus mirabidis, Proteus penneri. Protochlamydia naegleriophila, Pseudomonadota, Pseudomonas spp., Pseudomonas aeruginosa, Pseudomonas luteola, Pseudomonas teessidea, Pseudoxanthomonas broegbernensis, Pseudoxanthomonas japonensis, Raineya orbicola, Reichenbachiellaceae, Rickettsia parkeri, Rickettsia, Salinibacter ruber, Salinicola halophilus, Salmonella spp., Salmonella bongori, Salmonella enterica, Samsonia, Saprospirales, Selenomonadales, Serratia marcescens, Shigella, Shimwellia, Solobacterium moorei, Sorangium cellulosum, Sphaerotilus natans, Sphingobacterium olei, Sphingomonas gei. Sphingosinicella humi, Spirochaela, Spirochaetaceae. Spirochaelales, Spirosomaceae, Sporomusa, Stenotrophomonas spp., Stenotrophomonas nitritireducens, Thalassolitnus marinus. Thermotoga neapolitana. Thorselliaceae, Treponemataceae, Vampirococcus, Verminephrobacter, Vibrio adaptatus, Tibrio aerogenes, Vibrio azasii, Vibrio campbellii, and Vibrio cholerae. In some examples, the gram-negative bacteria can comprise Lsscherichia coli (E. coli), Salmonella spp., Shigella, Pseudomonas spp., Moraxella spp., Helicobacter spp., Stenotrophomonas, Bdellovibrio, Legionella spp., or a combination thereof. In some examples, the gram-negative bacteria can comprise Neisseria gonorrhoeae, Neisseria meningitidis, Moraxella catarrhalis, Haemophilus influenzae, Klebsiella pneumoniae, Legionella pneumophila, Pseudomonas aeruginosa, Proteus mirabilis, Enterobacter cloacae, Serratia marcescens, Helicobacter pylori, Salmonella enteritidis, Salmonella typhi, Acinetobacter baumannii, or a combination thereof. In some examples, the gram-negative bacteria can comprise Escherichia coli, Pseudomonas aeruginosa, Chiamydia trachomatis, Yersinia pestis, or a combination thereof.

In some examples, the biological amphiphile comprises an endotoxin secreted by Escherichia coli.

### Methods of Making

Also disclosed herein are methods of making any of the assays disclosed herein. For example, the methods can comprise disposing the lubricant on the ligand-modified substrate, for example using printing, spin coating, drop-casting, zone casting, dip coating, blade coating, spraying, vacuum filtration, or combinations thereof.

In some examples, the methods can further comprise making the ligand-modified substrate.

In some examples, the methods can comprise contacting the substrate with a plurality of ligands having a functional group configured to covalently or ionically bond to the substrate, such as a silyl, thiol (—SH), carboxyl (—COOH), or amine (—NH) group.

### Methods of Use

Also disclosed herein are methods of use of any of the assays disclosed herein.

For example, also disclosed herein are methods comprising: contacting any of the assays disclosed herein with a liquid sample; subsequently tilting the assay; and subsequently visually inspecting the liquid sample disposed on the assay to determine a property of the liquid sample.

The liquid sample can comprise any liquid sample of interest. In some examples, the liquid sample can comprise a solvent. The solvent can, for example, comprise tetrahydrofuran (THF), N-methyl-2-pyrrolidone (NMP), dimethylformamide (DMF), N-methylformamide, formamide, dichloromethane (CH₂Cl₂), ethylene glycol, polyethylene glycol, glycerol, alkane diol, ethanol, methanol, propanol, isopropanol, water, acetonitrile, chloroform, toluene, methyl acetate, ethyl acetate, acetone, hexane, heptane, tetraglyme, propylene carbonate, diglyme, dimethyl sulfoxide (DMSO), dimethoxyethane, xylene, dimethylacetamide, or combinations thereof. In some examples, solvent comprises water, such that the liquid sample comprises an aqueous solution.

In some examples, the methods can further comprise collecting the liquid sample. In some examples, the liquid sample is collected in a vial.

In some examples, the methods can further comprise purifying or treating the liquid sample before contacting the liquid sample with the assay. Purifying the liquid sample can, for example, comprise filtering, centrifuging, electrophoresis, extraction, or a combination thereof. Treating the liquid sample can, for example, comprise neutralization, buffer exchange, or a combination thereof.

In some examples, visually inspecting the liquid sample disposed on the assay can comprise direct visual inspection, e.g. naked-eye inspection. In some examples, visually inspecting the liquid sample disposed on the assay can comprise examining a picture of video of the liquid sample disposed on the assay.

In some examples, visually inspecting the liquid sample disposed on the assay can comprise comprises determining the angle at which the liquid sample starts to slide, the speed at which the liquid sample moves along the assay when tilted at a given angle, the distance that the liquid sample moves along the assay when tilted at a given angle, or a combination thereof.

The property of the liquid sample can, for example, comprise the presence of the amphiphile the liquid sample, the concentration of the amphiphile in the liquid sample, the identity of the amphiphile, or a combination thereof.

In some examples, the amphiphile comprises a biological amphiphile. In some examples, the biological amphiphile comprises an endotoxin. In some examples, the biological amphiphile comprises an endotoxin secreted by a gram-negative bacteria. In some examples, the biological amphiphile comprises an endotoxin secreted by Escherichia coli.

In some examples, the amphiphile is present in the liquid sample at a concentration of 1 picogram (pg)/mL or more (e.g., 2 pg/mL or more, 3 pg/mL or more, 4 pg/mL or more, 5 pg/mL or more, 10 pg/mL or more, 15 pg/mL or more, 20 pg/mL or more, 25 pg/mL or more, 30 pg/mL or more, 35 pg/mL or more, 40 pg/mL or more, 45 pg/mL or more, 50 pg/mL or more, 60 pg/mL or more, 70 pg/mL or more, 80 pg/mL or more, 90 pg/mL or more, 100 pg/mL or more, 125 pg/mL or more, 150 pg/mL or more, 175 pg/mL or more, 200 pg/mL or more, 225 pg/mL or more, 250 pg/mL or more, 300 pg/mL or more, 350 pg/mL or more, 400 pg/mL or more, 450 pg/mL or more, 500 pg/mL or more, 600 pg/mL or more, 700 pg/mL or more, 800 pg/mL or more, 900 pg/mL or more, 1 nanogram (ng)/mL or more, 1.25 ng/mL or more, 1.5 ng/mL or more, 1.75 ng/mL or more, 2.25 ng/mL or more, 2 ng/mL or more, 2.5 ng/mL or more, 3 ng/mL or more, 3.5 ng/mL or more, 4 ng/mL or more, 4.5 ng/mL or more, 5 ng/mL or more, 6 ng/mL or more, 7 ng/mL or more, 8 ng/mL or more, 9 ng/mL or more, 10 ng/mL or more, 15 ng/mL or more, 20 ng/mL or more, 25 ng/mL or more, 30 ng/mL or more, 35 ng/mL or more, or 40 ng/mL or more). In some examples, the amphiphile is present in the liquid sample at a concentration of 50 nanograms (ng)/mL or less (e.g., 45 ng/mL or less, 40 ng/mL or less, 35 ng/mL or less, 30 ng/mL or less, 25 ng/mL or less, 20 ng/mL or less, 15 ng/mL or less, 10 ng/mL or less, 9 ng/mL or less, 8 ng/mL or less, 7 ng/mL or less, 6 ng/mL or less, 5 ng/mL or less, 4.5 ng/mL or less, 4 ng/mL or less, 3.5 ng/mL or less, 3 ng/mL or less, 2.5 ng/mL or less, 2.25 ng/mL or less, 2 ng/mL or less, 1.75 ng/mL or less, 1.5 ng/mL or less, 1.25 ng/mL or less, 1 ng/mL or less, 900 picograms (pg)/mL or less, 800 pg/mL or less, 700 pg/mL or less, 600 pg/mL or less, 500 pg/mL or less, 450 pg/mL or less, 400 pg/mL or less, 350 pg/mL or less, 300 pg/mL or less, 250 pg/mL or less, 225 pg/mL or less, 200 pg/mL or less, 175 pg/mL or less, 150 pg/mL or less, 125 pg/mL or less, 100 pg/mL or less, 90 pg/mL or less, 80 pg/mL or less, 70 pg/mL or less, 60 pg/mL or less, 50 pg/mL or less, 45 pg/mL or less, 40 pg/mL or less, 35 pg/mL or less, 30 pg/mL or less, 25 pg/mL or less, 20 pg/mL or less, 15 pg/mL or less, 10 pg/mL or less, or 5 pg/mL or less). The concentration of the amphiphile in the liquid sample can range from any of the minimum values described above to any of the maximum values described above. For example, the amphiphile can be present in the liquid sample at a concentration of from 1 picogram/mL to 50 nanograms/mL (e.g., from 1 pg/mL to 100 pg/mL, from 100 pg/mL to 50 ng/mL, from 1 pg/mL to 10 pg/mL, from 10 pg/mL to 100 pg/mL, from 100 pg/mL to 1 ng/mL, from 1 ng/mL to 50 ng/mL, from 5 pg/mL to 50 ng/mL, from 1 pg/mL to 45 ng/mL, from 5 ng/mL to 45 ng/mL, from 25 pg/mL to 150 pg/mL, from 1 ng/mL to 50 ng/mL, or from 100 picograms/mL to 1 nanogram/mL).

In some examples, the liquid sample comprises a droplet. In some examples, the liquid sample has a volume of 1 microliter (μL) or more (e.g., 2 μL or more, 3 μL or more, 4 μL or more, 5 μL or more, 6 μL or more, 7 μL or more, 8 μL or more, 9 μL or more, 10 μL or more, 15 μL or more, 20 μL or more, 25 μL or more, 30 μL or more, 35 μL or more, 40 μL or more, 45 μL or more, 50 μL or more, 60 μL or more, 70 μL or more, 80 μL or more, 90 μL or more, 100 μL or more, 125 μL or more, 150 μL or more, 175 μL or more, 200 μL or more, 225 μL or more, 250 μL or more, 300 μL or more, 350 μL or more, 400 μL or more, 450 μL or more, 500 μL or more, 600 μL or more, 700 μL or more, 800 μL or more, 900 μL or more, 1 milliliter (mL) or more, 1.25 mL or more, 1.5 mL or more, 1.75 mL or more, 2 mL or more, 2.25 mL or more, 2.5 mL or more, 3 mL or more, 3.5 mL or more, 4 mL or more, 4.5 mL or more, 5 mL or more, 6 mL or more, 7 mL or more, 8 mL or more, 9 mL or more, 10 mL or more, 15 mL or more, 20 mL or more, 25 mL or more, 30 mL or more, 35 mL or more, or 40 mL or more). In some examples, the liquid sample has a volume of 50 milliliters (mL) or less (e.g., 45 mL or less, 40 mL or less, 35 mL or less, 30 mL or less, 25 mL or less, 20 mL or less, 15 mL or less, 10 mL or less, 9 mL or less, 8 mL or less, 7 mL or less, 6 mL or less, 5 mL or less, 4.5 mL or less, 4 mL or less, 3.5 mL or less, 3 mL or less, 2.5 mL or less, 2.25 mL or less, 1.75 mL or less, 1.5 mL or less, 1.25 mL or less, 1 mL or less, 900 μL or less, 800 μL or less, 700 μL or less, 600 μL or less, 500 μL or less, 450 μL or less, 400 μL or less, 350 μL or less, 300 μL or less, 250 μL or less, 225 μL or less, 200 μL or less, 175 μL or less, 150 μL or less, 125 μL or less, 100 μL or less, 90 μL or less, 80 μL or less, 70 μL or less, 60 μL or less, 50 μL or less, 45 μL or less, 40 μL or less, 35 μL or less, 30 μL or less, 25 μL or less, 20 μL or less, 15 μL or less, 10 μL or less, 9 μL or less, 8 μL or less, 7 μL or less, 6 μL or less, or 5 μL or less). The volume of the liquid sample can range from any of the minimum values described above to any of the maximum values described above. For example, the liquid sample can have a volume of from 1 microliter (μL) to 50 milliliters (mL) (e.g., from 1 μL to 100 μL, from 100 μL to 50 mL, rom 1 μL to 10 μL, from 10 μL to 100 μL, from 100 μL to 1 mL, from 1 mL to 50 mL, from 5 μL to 50 mL, from 1 μL to 45 mL, or from 5 μL to 45 mL).

In some examples, the methods can further comprise diagnosing and/or monitoring an infection with the biological amphiphile in a subject based on the property of the liquid sample. In some examples, the methods can further comprise selecting a course of therapy for the subject based on the property of the liquid sample.

Any of the methods disclosed herein can be carried out in whole or in part on one or more computing or processing devices. In certain examples, the methods can be carried out in whole or in part on a computing device comprising a processor and a memory operably coupled to the processor, the memory having further computer-executable instructions stored thereon that, when executed by the processor, cause the processor to carry out one or more of the method steps described above.

### Devices

Also disclosed herein are devices, for example for performing any of the methods described herein.

For example, also disclosed herein are devices comprising: a receptacle configured to at least partially contain any of the assays disclosed herein; wherein, when the device is assembled together with a liquid sample, then the receptacle is configured to at least partially contain the assay in contact with the liquid sample.

In some examples, the device can further comprise a means for tilting the assay to a desired angle, such as a movable and/or motorized stage. In some examples, the device can be a handheld device, and the means for tilting the assay can comprise using a hand of a user.

In some examples, the device can further comprise a detector configured to collect an optical signal from the liquid sample disposed on the assay. For example, the detector can comprise a camera configured to take a picture and/or video of the assay and/or liquid sample. In some examples, the camera can be configured to take a picture and/or video of the assay before, after, and/or during any of the methods od use disclosed herein, for example such that the visual inspection can be performed remotely in time and/or space.

In some examples, the device is further configured to output the property of the liquid sample and/or a feedback signal based on the property of the liquid sample. In some examples, the device can further comprise one or more output devices (e.g., a display, speakers, printer, LED, etc.) configured to output the property of the liquid sample and/or a feedback signal based on the property of the liquid sample. The feedback signal can, for example, comprise haptic feedback, auditory feedback, visual feedback, or a combination thereof.

In some examples, the device is a point-of-care device. In some examples, the device is a handheld device. In some examples, the device is a benchtop device. In some examples, the device is a high-throughput device. For example, the device can be a high-throughput device configured to analyze a plurality of liquid samples.

In some examples, the device can further comprise a computing device configured to receive and process the optical signal from the detector to determine a property of the liquid sample.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1

Amphiphiles are molecules composed of both polar and apolar moieties. Amphiphiles can self-assemble at interfaces and can lead to measurable changes to surface properties at concentrations close to their critical micelle concentrations (CMCs). Biological amphiphiles can affect ecological and/or biological functions even at extremely low concentrations. Therefore, the detection of low amphiphile concentrations can be used to monitor the condition of such systems.

Reliable and sensitive detection of various biological amphiphiles (e.g., endotoxin secreted by gram-negative bacteria such as Escherichia coli) can provide important safety information, such as for water and food supplies. Endotoxins are large lipopolysaccharides consisting of a lipid A covalently bonded to a high molecular weight polysaccharide segment.

Endotoxins can be an indicator of bacterial contamination. For instance, consumption of water which contains endotoxins at concentrations as low as 2 ng/mL may induce sepsis in humans (Rapala J et al. Water Res. 2002, 36, 2627-2635; Cohen J. Nature. 2002, 420, 885-891). Representative concentrations of endotoxins for various health-related contexts are shown in Table 1. In general, the concentration range of interest for endotoxin characterization is pg/mL to ng/mL.

TABLE 1
Representative concentrations of endotoxins
for various health-related contexts.

| Category | Subject | Endotoxin Concentration |
|---|---|---|
| Clinical | ICU admitted persons, not | <157 ± 140 pg/mL in |
| Relevance | with diagnosis of sepsis | blood |
| | Patients with sepsis | 470 ± 57 pg/mL in blood |
| | Lethal | >ng/mL in blood |
| Medical | Human pyrogenic dose | *Non-intrathecal: <500 pg/ |
| Device and | | kg |
| Pharmaceutical | | *Intrathecal |
| Manufacturing | | administration: <20 pg/kg |
| | Sterile water for injection | 25 pg/mL |
| | Sterile water for irrigation | 25 pg/mL |
| | Water for inhalation | 50 pg/mL |
| Public/ | Background in air | 100 pg/m³ of air |
| Occupational | Levels in air for acute | 200 ng/m³ of air |
| Health | bronchoconstriction and | |
| | chronic inflammation | |
| Food Safety | Milk sterilized by LTLT | 27 pg/mL |
| | Yogurt | 5-50 ng/mL |

However, monitoring the concentration of endotoxins is conventionally achieved using assays that rely upon specialized chemicals and equipment. For example, the concentration of endotoxins can be monitored using assays based on limulus amebocyte lysate (LAL), liquid crystals, and biological fluorescent proteins. The limulus amebocyte lysate (LAL) assay relies upon blood extracted from horseshoe crab, posing both ecological and ethical concerns. Because these assays rely upon specialized chemicals and equipment, they are unsuitable for portable and fast in-situ or point-of-care amphiphile detection.

Herein, a simple, low-cost, naked eye method for detection of amphiphiles, such as endotoxins, in aqueous solution/droplets is developed. Instead of involving silicon wafers and complex microfabrication procedure, a method of synthesis of ligand-modified glass substrates is proposed. A goal is to sense the concentration of amphiphiles (e.g., endotoxins) and/or sort droplets based on amphiphile (e.g., endotoxin) concentration using a simple procedure that does not involve complex equipment.

### Methods:

Silane functionalization. Glass slides were placed in 1% v/v heptane solution containing trichloro(octadecyl)silane and octyltrichlorosilane (1:1 molar ratio) for 40 minutes. After functionalization, the slide was rinsed using dichloromethane and dried by nitrogen gas.

Coating of silicone oil on silane-functionalized glass substrates. Silicone oil (10 cSt) was coated on silane-functionalized glass substrates by spin-coating at 350 rpm for 4.5 minutes.

Results. As the concentration of endotoxin increased, the static pinning force of endotoxin-containing aqueous droplets on the silicone oil-coated, silane-functionalized glass substrates increased. Compared with the sensitivity of microstructured silicon wafer for detection of endotoxins (˜1 ng/mL or ˜5-10 EU/mL for endotoxin detection), the silicone oil-coated, silane-functionalized glass substrates exhibit a sensitivity of ˜30 nM for SDS and ˜100 pg/mL for endotoxin, which corresponds to ˜0.5-1 EU/mL of endotoxin. This enhancement in the sensitivity is caused by the increase in the total contact angle length of aqueous droplets on the surface, which can broaden the practical application of these surfaces.

In some examples, the silane-functionalized glass substrate can be configured to be able to sort droplets based on the concentration of the amphiphile and/or the volume of the droplet.

Optimization of surface chemical modification and surface topography can further improve sensitivity of amphiphile detection.

The use paper/plastic as low-cost, flexible substrates for naked-eye detection of amphiphiles can also be explored.

### Example 2

Described herein are compositions, devices, systems, and methods for detection of an amphiphile using visual inspection of a ligand-modified substrate.

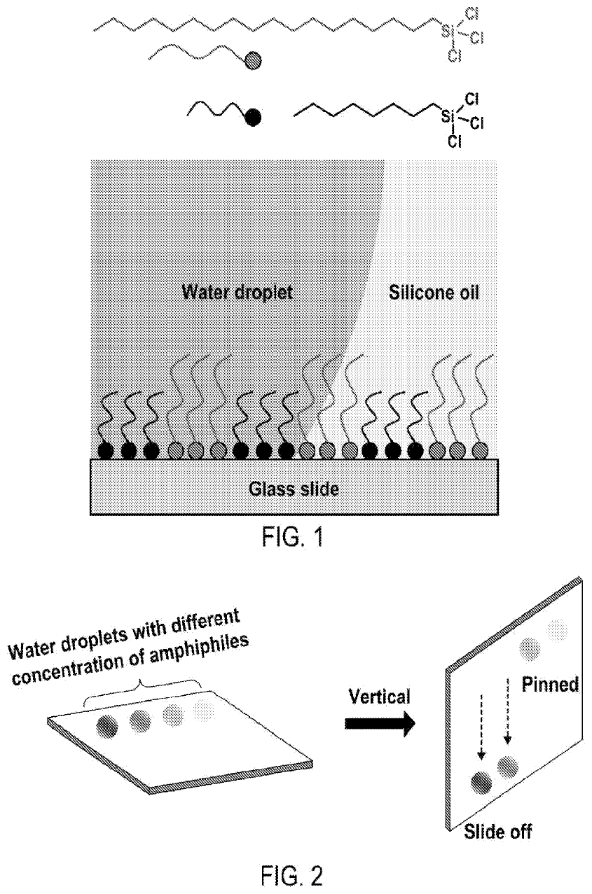

In some examples, a mixture of at least two types of silane molecules with different molecular sizes (e.g., length) can be used to functionalize glass surfaces. This mixture can create mis-packing of molecules and thus nanoscale roughness on the surface. When placing a water droplet on the surface, the amphiphiles in the water droplet can further assemble at the silane-coated glass substrates, resulting in droplet mobility changes compared with pure water droplet or droplets with different concentration of amphiphiles (FIG. 1-FIG. 2).

In terms of practical applications, there are several advantages. First, micrometer-sized or sub-micrometer-sized topography on the surfaces such as pillars or colloidal particles are not needed. Second, this sensor is very simple—it does not need any complicated equipment.

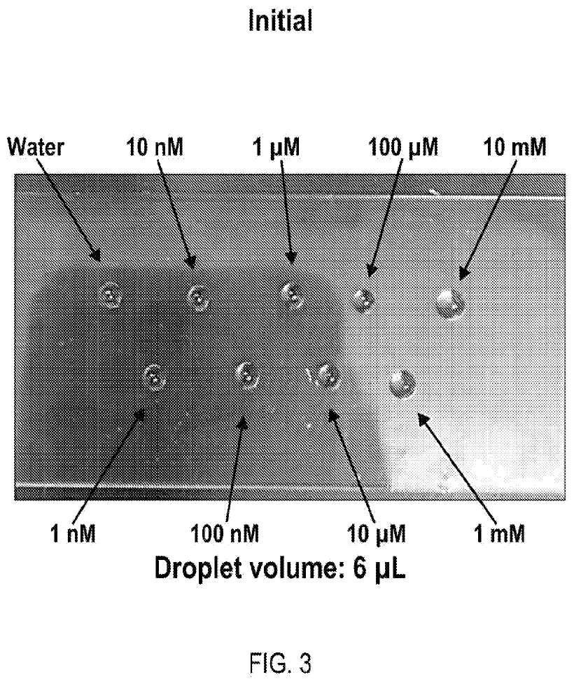

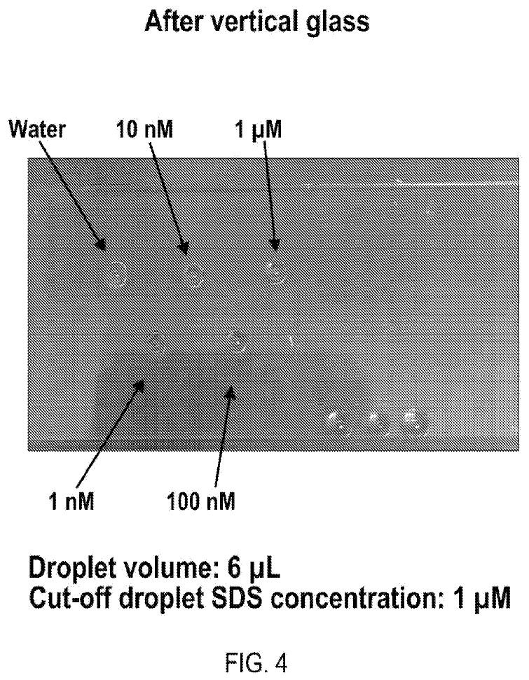

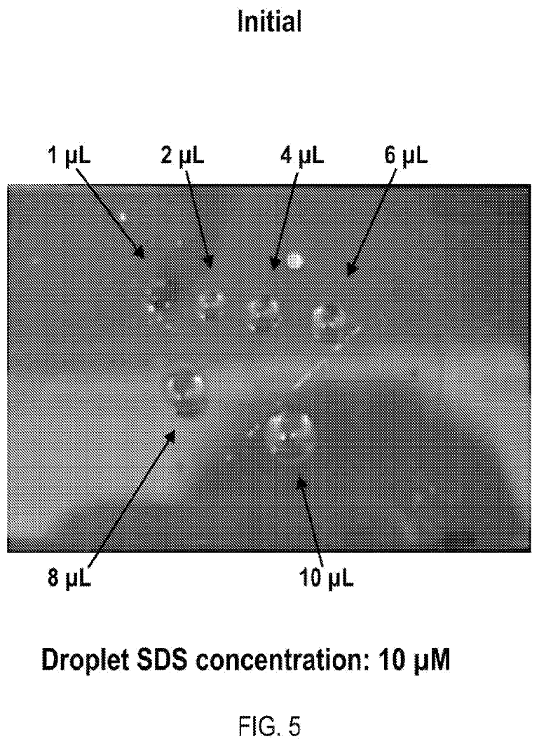

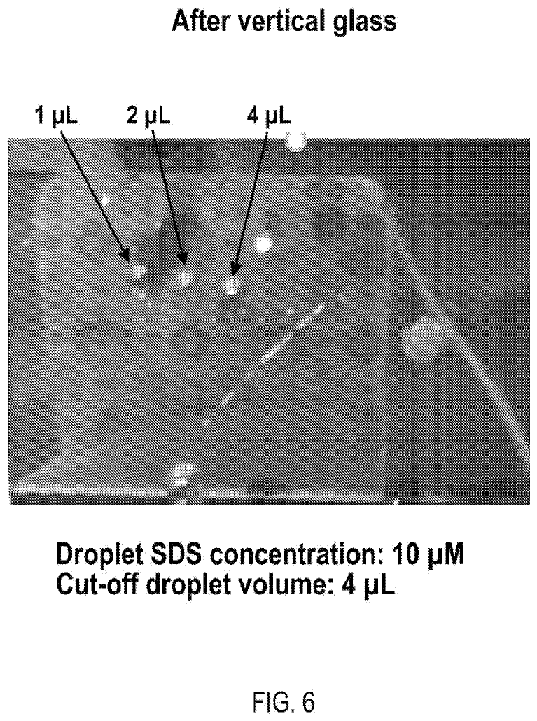

In previous work, a goniometer was needed to precisely control the tilting of the surfaces. In this design, the surface tilting does not need such precise control, the surface can simply be tilted to roughly vertical to sort droplets based on the concentration of the amphiphile and/or the volume of the droplet. As shown Table 2 and FIG. 3-FIG. 6, a series of droplets with different volumes were placed on the sensor surface and then the surface was titled to roughly be vertical, and it was observed which threshold water droplet volume and/or amphiphile concentration remained pinned on the surface.

TABLE 2
Droplet sorting results.

| SDS | | Droplet Volume | | | | | |
|---|---|---|---|---|---|---|---|
| Concentration | | 1 μL | 2 μL | 4 μL | 6 μL | 8 μL | 10 μL |
| Water | | Pinned | Pinned | Pinned | Pinned | Pinned | Slide |
| 1 | nM | Pinned | Pinned | Pinned | Pinned | Pinned | Slide |
| 10 | nM | Pinned | Pinned | Pinned | Pinned | Pinned | Slide |
| 100 | nM | Pinned | Pinned | Pinned | Pinned | Pinned | Slide |
| 1 | μM | Pinned | Pinned | Pinned | Pinned | Slide | Slide |
| 10 | μM | Pinned | Pinned | Pinned | Slide | Slide | Slide |
| 100 | μM | Pinned | Pinned | Slide | Slide | Slide | Slide |
| 1 | mM | Pinned | Slide | Slide | Slide | Slide | Slide |
| 10 | mM | Slide | Slide | Slide | Slide | Slide | Slide |

### Exemplary Aspects

In view of the described compositions, devices, systems and methods for detection of an amphiphile, herein below are described certain more particularly described aspects of the inventions. The particularly recited aspects should not, however, be interpreted to have any limiting effect on any different claims containing different or more general teachings described herein or that the “particular” aspects are somehow limited in some way other than the inherent meanings of the language and formulas literally used therein.

Example 1: An assay for detection of an amphiphile via visual inspection, the assay comprising: a ligand-modified substrate comprising a substrate having a plurality of ligands attached to a surface thereof; and a lubricant disposed on the ligand-modified substrate, such that the lubricant forms a film on the ligand-modified substrate; wherein the plurality of ligands comprises a first population of ligands and a second population of ligands, the first population of ligands being different than the second population of ligands.

Example 2: The assay of any examples herein, particularly example 1, wherein the substrate comprises silicon, glass, a polymer, a cellulosic substrate, a metal, or a combination thereof.

Example 3: The assay of any examples herein, particularly example 1 or example 2, wherein the substrate comprises glass, such as a glass slide.

Example 4: The assay of any examples herein, particularly examples 1-3, wherein the substrate comprises a flexible substrate.

Example 5: The assay of any examples herein, particularly examples 1-4, wherein the substrate comprises a rigid substrate.

Example 6: The assay of any examples herein, particularly examples 1-5, wherein each of the plurality of ligands comprises a silyl, a thiol, a sulfate, a carboxylic acid, an amine, or a combination thereof.

Example 7: The assay of any examples herein, particularly examples 1-6, wherein each of the plurality of ligands comprises a silyl, such as a silane or an alkoxy silane.

Example 8: The assay of any examples herein, particularly examples 1-7, wherein the first population of ligands have a first average length, the second populations of ligands have a second average length, and the first average length is different than the second average length.

Example 9: The assay of any examples herein, particularly examples 1-8, wherein the lubricant comprises silicon oil.

Example 10: The assay of any examples herein, particularly examples 1-9, wherein the amphiphile comprises a biological amphiphile.

Example 11: The assay of any examples herein, particularly example 10, wherein the biological amphiphile comprises an endotoxin.

Example 12: The assay of any examples herein, particularly examples 10-11, wherein the biological amphiphile comprises an endotoxin secreted by a gram-negative bacteria.

Example 13: The assay of any examples herein, particularly examples 10-12, wherein the biological amphiphile comprises an endotoxin secreted by Escherichia coli.

Example 14: The assay of any examples herein, particularly examples 1-13, wherein the assay comprises a plurality of domains, wherein each domain is different with respect to ligand functionalization (e.g., identity of ligand, density or concentration of ligands).

Example 15: A method of making the assay of any examples herein, particularly examples 1-14, the method comprising disposing the lubricant on the ligand-modified substrate.

Example 16: The method of any examples herein, particularly example 15, wherein the method further comprises making the ligand-modified substrate.

Example 17: The method of any examples herein, particularly example 16, wherein the method comprises contacting the substrate with a plurality of ligands having a functional group configured to covalently or ionically bond to the substrate.

Example 18: A method comprising: contacting the assay of any examples herein, particularly examples 1-14 with a liquid sample; subsequently tilting the assay; and subsequently visually inspecting the liquid sample disposed on the assay to determine a property of the liquid sample.

Example 19: The method of any examples herein, particularly example 18, wherein the liquid sample comprises a solvent.

Example 20: The method of any examples herein, particularly example 19, wherein the solvent comprises water.

Example 21: The method of any examples herein, particularly examples 18-20, wherein the method further comprises collecting the liquid sample.

Example 22: The method of any examples herein, particularly examples 18-21, wherein the method further comprises purifying the liquid sample before contacting the liquid sample with the assay.

Example 23: The method of any examples herein, particularly examples 18-22, wherein visually inspecting the liquid sample disposed on the assay comprises determining the angle at which the liquid sample starts to slide, the speed at which the liquid sample moves along the assay when tilted at a given angle, the distance that the liquid sample moves along the assay when tilted at a given angle.

Example 24: The method of any examples herein, particularly examples 18-23, wherein the property of the liquid sample comprises the presence of the amphiphile the liquid sample, the concentration of the amphiphile in the liquid sample, the identity of the amphiphile, or a combination thereof.

Example 25: The method of any examples herein, particularly examples 18-24, wherein the amphiphile comprises a biological amphiphile.

Example 26: The method of any examples herein, particularly example 25, wherein the biological amphiphile comprises an endotoxin.

Example 27: The method of any examples herein, particularly examples 25-26, wherein the biological amphiphile comprises an endotoxin secreted by a gram-negative bacteria.

Example 28: The method of any examples herein, particularly examples 25-27, wherein the biological amphiphile comprises an endotoxin secreted by Escherichia coli.

Example 29: The method of any examples herein, particularly examples 26-28, wherein the endotoxin is present in the liquid sample at a concentration of from 1 picogram/mL to 50 nanograms/mL.

Example 30: The method of any examples herein, particularly examples 26-29, wherein the endotoxin is present in the liquid sample at a concentration of from 100 picograms/mL to 1 nanogram/mL.

Example 31: The method of any examples herein, particularly examples 18-30, wherein the liquid sample comprises a droplet.

Example 32: The method of any examples herein, particularly examples 18-31, wherein the liquid sample has a volume of from 1 microliter (μL) to 50 milliliters (mL).

Example 33: The method of any examples herein, particularly examples 18-32, wherein visually inspecting the liquid sample disposed on the assay comprises naked-eye detection.

Example 34: The method of any examples herein, particularly examples 18-33, further comprising diagnosing and/or monitoring an infection with the biological amphiphile in a subject based on the property of the liquid sample.

Example 35: The method of any examples herein, particularly example 34, further comprising selecting a course of therapy for the subject based on the property of the liquid sample.

Example 36: A device comprising: a receptacle configured to at least partially contain the assay of any examples herein, particularly examples 1-14; wherein, when the device is assembled together with a liquid sample, then the receptacle is configured to at least partially contain the assay in contact with the liquid sample.

Example 37: The device of any examples herein, particularly example 36, further comprising a means for tilting the assay to a desired angle.

Example 38: The device of any examples herein, particularly examples 36-37, further comprising a detector configured to collect an optical signal from the liquid sample disposed on the assay.

Example 39: The device of any examples herein, particularly example 38, wherein the detector comprises a camera configured to take a picture and/or video of the assay and/or liquid sample.

Example 40: The device of any examples herein, particularly example 39, wherein the camera is configured to take a picture and/or video of the assay before, after, and/or during the method of any examples herein, particularly examples 24-40, for example such that the visual inspection can be performed remotely in time and/or space.

Example 41: The device of any examples herein, particularly examples 38-40, further comprising a computing device configured to receive and process the optical signal from the detector to determine a property of the liquid sample.

Example 42: The device of any examples herein, particularly example 41, wherein the device is further configured to output the property of the liquid sample and/or a feedback signal based on the property of the liquid sample.

Example 43: The device of any examples herein, particularly example 42, wherein the feedback signal comprises haptic feedback, auditory feedback, visual feedback, or a combination thereof.

Example 44: The device of any examples herein, particularly examples 36-43, wherein the device is a point-of-care device.

Example 45: The device of any examples herein, particularly examples 36-44, wherein the device is a handheld device.

Example 46: The device of any examples herein, particularly examples 36-44, wherein the device is a benchtop device.

Example 47: The device of any examples herein, particularly examples 36-44, wherein the device is a high-throughput device.

Example 48: The device of any examples herein, particularly example 47, wherein the device is configured to analyze a plurality of liquid samples.

Example 49: The device of any examples herein, particularly examples 36-48, wherein the device is configured to perform the method of any examples herein, particularly examples 18-35.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. An assay for detection of an amphiphile via visual inspection, the assay comprising:
a ligand-modified substrate comprising a substrate having a plurality of ligands attached to a surface thereof, and
a lubricant disposed on the ligand-modified substrate, such that the lubricant forms a film on the ligand-modified substrate;
wherein the plurality of ligands comprises a first population of ligands and a second population of ligands, the first population of ligands being different than the second population of ligands.

2. The assay of claim 1, wherein the substrate comprises silicon, glass, a polymer, a cellulosic substrate, a metal, or a combination thereof.

3. (canceled)

4. (canceled)

5. (canceled)

6. The assay of claim 1, wherein each of the plurality of ligands comprises a silyl, a thiol, a sulfate, a carboxylic acid, an amine, or a combination thereof.

7. The assay of claim 1, wherein each of the plurality of ligands comprises a silyl, such as a silane or an alkoxy silane.

8. The assay of claim 1, wherein the first population of ligands have a first average length, the second population of ligands have a second average length, and the first average length is different than the second average length.

9. The assay of claim 1, wherein the lubricant comprises silicon oil.

10. The assay of claim 1, wherein the amphiphile comprises a biological amphiphile.

11. The assay of claim 10, wherein the biological amphiphile comprises an endotoxin.

12. The assay of claim 11, wherein the biological amphiphile comprises an endotoxin secreted by a gram-negative bacteria.

13. (canceled)

14. The assay of claim 1, wherein the assay comprises a plurality of domains, wherein each domain is different with respect to ligand functionalization.

15. A method of making the assay of claim 1, the method comprising disposing the lubricant on the ligand-modified substrate.

16. (canceled)

17. (canceled)

18. A method comprising:
contacting the assay of claim 1 with a liquid sample;
subsequently tilting the assay; and
subsequently visually inspecting the liquid sample disposed on the assay to determine a property of the liquid sample.

19. (canceled)

20. (canceled)

21. (canceled)

22. (canceled)

23. The method of claim 18, wherein visually inspecting the liquid sample disposed on the assay comprises determining the angle at which the liquid sample starts to slide, the speed at which the liquid sample moves along the assay when tilted at a given angle, the distance that the liquid sample moves along the assay when tilted at a given angle.

24. The method of claim 18, wherein the property of the liquid sample comprises the presence of the amphiphile the liquid sample, the concentration of the amphiphile in the liquid sample, the identity of the amphiphile, or a combination thereof.

25. (canceled)

26. (canceled)

27. (canceled)

28. (canceled)

29. The method of claim 18, wherein the amphiphile comprises an endotoxin and the endotoxin is present in the liquid sample at a concentration of from 1 picogram/mL to 50 nanograms/mL.

30. (canceled)

31. The method of claim 18, wherein the liquid sample comprises a droplet.

32. (canceled)

33. The method of claim 18, wherein visually inspecting the liquid sample disposed on the assay comprises naked-eye detection.

34. The method of claim 18, wherein the amphiphile comprises a biological amphiphile, and the method further comprises diagnosing and/or monitoring an infection with the biological amphiphile in a subject based on the property of the liquid sample.

35. The method of claim 34, further comprising selecting a course of therapy for the subject based on the property of the liquid sample.

36. A device comprising:
a receptacle configured to at least partially contain the assay of claim 1;
wherein, when the device is assembled together with a liquid sample, then the receptacle is configured to at least partially contain the assay in contact with the liquid sample.

37.-49. (canceled)
